(19) United States
(12) Patent Application Publication
(10) Pub. No.: US 2020/0054721 A1
(43) Pub. Date: Feb. 20, 2020

(54) MATERIALS AND METHODS FOR TREATING OR PREVENTING GRAFT-VERSUS-HOST-DISEASE

(71) Applicants: Bruce R. BLAZAR, Minneapolis, CA (US); John H. GRIFFIN, La Jolla, CA (US); Ryan P. FLYNN, Minneapolis, MN (US); Ranjeet K. SINHA, La Jolla, CA (US); Regents of the University of Minnesota, Minneapolis, MN (US); The Scripps Research Institute, La Jolla, CA (US)

(72) Inventors: Bruce R. Blazar, Golden Valley, MN (US); John H. Griffin, La Jolla, CA (US); Ryan P. Flynn, Minneapolis, MN (US); Ranjeet K. Sinha, La Jolla, CA (US)

(21) Appl. No.: 16/343,640

(22) PCT Filed: Oct. 20, 2017
(86) PCT No.: PCT/US2017/057589

§ 371 Date: Apr. 19, 2019

Related U.S. Application Data
(60) Provisional application No. 62/411,081, filed on Oct. 21, 2016.

(51) Int. Cl. A61K 38/48; A61K 9/00; A61P 37/06

(52) U.S. Cl. CPC A61K 38/4866; A61P 37/06; A61K 9/0019

(57) ABSTRACT

This disclosure provides materials and methods for treating or preventing graft-versus-host-disease (GVHD) using protein C or activated protein C or a signaling-selective variant or mutant thereof.

### CROSS REFERENCE TO RELATED APPLICATIONS

This application claims the benefit of priority under 35 U.S.C. § 119(e) to U.S. Application No. 62/411,081 filed Oct. 21, 2016.

### FEDERALLY SPONSORED RESEARCH OR DEVELOPMENT

This invention was made with government support under AI 056299, HL031950, and HL052246 awarded by the National Institutes of Health. The government has certain rights in the invention.

### TECHNICAL FIELD

This disclosure generally relates to materials and methods for treating or preventing graft-versus-host-disease (GVHD).

### BACKGROUND

Currently, therapies for graft-versus-host-disease (GVHD) are limited, and typically treat the symptoms as opposed to the actual disease. Accordingly, novel therapies for GVHD would be beneficial.

### SUMMARY

The methods described herein can reduce morbidity and mortality in subjects who undergo allogeneic hematopoietic stem-cell transplantation. As demonstrated herein, GVHD can be treated or prevented in subjects by using APC or signaling-selective variants or mutants of APC.

In one aspect, a method of treating or preventing graft-versus-host disease (GVHD) in a subject is provided. Such a method typically includes administering a therapeutically effective amount of protein C or a variant or mutant thereof to the subject.

In another aspect, a method of treating or preventing graft-versus-host disease (GVHD) in a subject is provided. Such a method typically includes contacting donor T cells with a therapeutically effective amount of protein C or a variant or mutant thereof.

In some embodiments of the methods described herein, the GVHD is a result of a hematopoietic stem cell transplant (e.g., an allogeneic hematopoietic stem-cell transplant). In some embodiments of the methods described herein, the hematopoietic stem cell transplant is a bone marrow transplant (e.g., an allogeneic bone marrow transplant). The GVHD can be chronic GVHD or acute GVHD. In some embodiments of the methods described herein, the subject suffers from fibrosis and/or multi-organ system disease with bronchiolitis obliterans (BO).

In some embodiments of the methods described herein, the protein C or variant or mutant thereof is an activated protein C (APC) or variant or mutant thereof. In some embodiments of the methods described herein, the APC variant or mutant thereof is a signaling-selective variant or mutant. In some embodiments of the methods described herein, the APC variant or mutant is 3A-APC (APC w/Lys191-193Ala mutation). In some embodiments of the methods described herein, the APC variant or mutant is 5A-APC (APC w/Lys191-193Ala and Arg229-230Ala mutations).

In some embodiments of the methods described herein, the protein C or variant or mutant thereof is administered at least once a day. In some embodiments, the protein C or variant or mutant thereof is administered to the subject at a dose of about 0.01 mg to about 0.6 mg (e.g., about 0.12 mg to about 0.24 mg) of the protein C or variant or mutant thereof per kilogram (kg) of the subject. In some embodiments of the methods described herein, the therapeutically-effective amount of the protein C or variant or mutant thereof is about 0.01 mg to about 0.6 mg (e.g., about 0.12 mg to about 0.24 mg) of the protein C or variant or mutant thereof per kilogram (kg) of the subject.

In some embodiments of the methods described herein, the protein C or variant or mutant thereof is administered intraperitoneally. In some embodiments of the methods described herein, the administering is initiated prior to the subject receiving a bone marrow transplant, coincidentally with the subject receiving a bone marrow transplant, and/or after the subject has received a bone marrow transplant.

In some embodiments of the methods described herein, the donor T cells are in blood or in bone marrow. In some embodiments of the methods described herein, the donor T cells are contacted with the protein C or variant or mutant thereof ex vivo.

In some embodiments of the methods described herein, GVHD is treated in the subject when the GVHD or one or more symptoms associated with the GVHD is reversed, alleviated or inhibited. In some embodiments of the methods described herein, GVHD is prevented in the subject when the GVHD or one or more symptoms associated with GVHD is avoided or precluded.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A is a graph showing that treatment with each of three different versions of activated protein C (“aPC” or “APC”) for 28 days improved lung function based on resistance tests. BM, bone marrow cells; S, splenocytes.

FIG. 1B is a graph showing that treatment with each of three different versions of activated protein C (“aPC” or “APC”) for 28 days improved lung function based on elastance tests. BM, bone marrow cells; S, splenocytes.

FIG. 1C is a graph showing that treatment with each of three different versions of activated protein C (“aPC” or “APC”) for 28 days improved lung function based on compliance tests. BM, bone marrow cells; S, splenocytes.

FIG. 2A is a graph showing that treatment with each of three different versions of APC significantly reduced the number of T follicular helper cell (CD4+CXCR5+PD1hi).

FIG. 2B is a graph showing that treatment with each of three different versions of APC significantly reduced splenic germinal center (GC) size.

FIG. 2C is a graph showing that treatment with each of three different versions of APC significantly reduced Th17 frequencies.

FIG. 3A is a graph showing that T cells transplanted from wild type (WT) mice and from mice carrying the mutation of either Arg41Gln (R41Q) or Arg46Gln (R46Q) in PAR1 (protease activated receptor 1 (murine gene F2r)) determine whether or not APC reduces damage due to GVHD based on resistance tests. Veh, control treatment without APC.

FIG. 3B is a graph showing that T cells transplanted from wild type (WT) mice and from mice carrying the mutation of either Arg41Gln (R41Q) or Arg46Gln (R46Q) in PAR1 (protease activated receptor 1 (murine gene F2r)) determine whether or not APC reduces damage due to GVHD based on elastance tests. Veh, control treatment without APC.

FIG. 3C is a graph showing that T cells transplanted from wild type (WT) mice and from mice carrying the mutation of either Arg41Gln (R41Q) or Arg46Gln (R46Q) in PAR1 (protease activated receptor 1 (murine gene F2r)) determine whether or not APC reduces damage due to GVHD based on compliance tests. Veh, control treatment without APC.

### DETAILED DESCRIPTION

Graft-versus-host disease (GVHD), and particularly, chronic graft-versus-host disease (cGVHD), is a significant cause of morbidity and mortality after hematopoietic stem cell transplantation, particularly after allogeneic hematopoietic stem cell transplantation.

This disclosure demonstrates that treatment with protein C, or activated protein C (APC), can significantly reduce the damage, e.g., to cells and tissues, caused by GVHD. As demonstrated herein, daily treatment with APC (e.g., recombinant APC) was highly effective in treating ongoing cGVHD in an animal model. Given the safety and efficacy record of APCs in clinical studies, these data strongly support the use of APCs (or variants or mutants thereof) to treat GVHD, and particularly cGVHD that has been refractory to other therapies (e.g., with bronchiolitis obliterans (BO)).

### Graft-Versus-Host Disease (GVHD)

Hematopoietic stem cell transplant (e.g., from a blood or bone marrow) from one individual to another, referred to as an allogeneic transplant (e.g., allogeneic hematopoietic stem cell transplant), can result in the recipient developing GVHD. Older individuals, individuals who have received a peripheral blood transplant (instead of a bone marrow transplant), and individuals who have received a transplant from a mismatched or unrelated donor have a greater risk of developing GVHD. In addition, individuals who have had acute GVHD (aGVHD) have a greater risk of developing cGVHD.

cGVHD can appear at any time after allogeneic transplant, from several months to several years after transplant. Typically, cGVHD begins later after transplant and lasts longer than aGVHD. cGVHD can occur in the skin (e.g., rash, raised, or discolored areas, skin thickening or tightening), liver (e.g., abdominal swelling, yellow discoloration of the skin and/or eyes, and abnormal blood test results), eyes (e.g., dry eyes or vision changes), gastrointestinal tract (e.g., mouth, esophagus, stomach, intestines) (e.g., dry mouth, white patches inside the mouth, pain or sensitivity, difficulty swallowing, pain with swallowing, or weight loss), lungs (e.g., shortness of breath or changes on chest X-rays), neuromuscular system (e.g., fatigue, muscle weakness, or pain), or genitourinary tract (e.g., increased frequency of urination, burning or bleeding with urination, vaginal dryness/tightening, or penile dysfunction), which can result in individuals presenting with a wide variety of symptoms. cGVHD is most often diagnosed by the presence of a skin rash or by changes in the eyes or mouth. cGVHD can cause damage in the glands that produce tears in the eyes and saliva in the mouth, resulting in dry eyes or a dry mouth, and individuals can have mouth ulcers, skin rashes, or liver inflammation. cGVHD also can result in formation of scar tissue in the skin (e.g., cutaneous sclerosis), and joints, and damage to air passages in the lungs, resulting in bronchiolitis obliterans (BO) syndrome and/or fibrosis. cGVHD also results in a significantly increased risk of the subject developing infections.

Following a blood or bone marrow stem cell transplant, individuals (also referred to as recipient subjects) can be administered one or more immunosuppressants (e.g., prophylactically) to lower the risk of developing GVHD. In addition, treatment options once a subject has been diagnosed with GVHD generally include administration of one or more immunosuppressants (e.g., a long-term immunosuppressive regimen). While immunosuppressants decrease the ability of donor T cells to initiate and maintain an immune response against the recipient, fungal, bacterial and viral infections are significant risks with any type of immunosuppressant regimen.

### Activated Protein C (APC) and Variants or Mutants Thereof

Protein C (CAS #146340-20-7) is a vitamin K-dependent glycoprotein that is structurally similar to other vitamin K-dependent proteins affecting blood clotting, such as Factor VII, Factor IX and Factor X. Synthesis of protein C begins in the liver with a single-chain precursor molecule (i.e., a 32 amino acid N-terminus signal peptide preceding a pro-peptide and the mature protein sequence). Following formation and secretion of protein C, the pro-peptide and a dipeptide of Lys198 and Arg199 typically is removed. The normal circulating molecule is a protease zymogen, protein C, which can be converted into an active protease, activated protein C (“APC” or “aPC”), by limited proteolysis that releases a small activation peptide. Overall, the mature protein C zymogen as well as APC includes one light chain (21 kDa) and one heavy chain (41 kDa) connected by a disulfide bond (between Cys183 and Cys319).

Activated Protein C (APC) is a trypsin-like plasma serine protease that exerts multiple beneficial pharmacological activities. Receptors on multiple cell types have been implicated in APCs cell signaling activities. In the current paradigm, endothelial cell protein C receptor (EPCR)-bound APC cleaves protease activated receptor (PAR)-1 at Arg46 to initiate arrestin-dependent biased signaling, which is cytoprotective, anti-inflammatory, able to alter gene expression profiles, and able to alter differentiation and development of cell lineages, while cleavage by thrombin in PAR1 at Arg41 initiates G-protein-dependent pro-inflammatory signaling. See, for example, Griffin et al. (2015, Blood, 125:2898-907); Griffin et al. (2016, Thrombosis Res., 141 Suppl 2:S62-64); Griffin et al. (2016, Atheroscler. Thromb. Vasc. Biol., in press); and Mosnier et al. (2012, Blood, 120:5237-46)).

APC is pleiotropic in its proteolytic actions, with two main classes of functions: anti-thrombotic activity and initiation of cell signaling. The activity of APC depends on whether or not APC interacts with one or more receptors on the surface of cells that are being targeted or interacts with its coagulation factor substrates, factors VIIIa and Va. While not wishing to be bound by any particular theory, the anti-thrombotic functions seem to occur when APC irreversibly proteolytically inactivates Factor Va and Factor VIIIa to produce Factor Vi and Factor VIIIi, respectively. On the other hand, the cell signaling functions seem to occur when APC, bound to endothelial protein C receptor (EPCR) on a variety of cell types, acts on the effector substrate, protease-activated receptor-1 (PAR-1). The experiments described herein (e.g., using signaling-selective APC mutants as well as donor T cells obtained from novel PAR1-genetically modified mice) suggest that the mechanism responsible for the observed suppression of GVHD results predominantly from the cell signaling activity of APC (i.e., the initiation of which is dependent upon PAR1 cleavage, specifically at Arg46) and not the anticoagulant activity of APC (i.e., the initiation of which is dependent upon PAR1 cleavage, specifically at Arg41).

APCs beneficial effects on endothelial, epithelial, and neuronal cells have been well described. While APC has been previously associated, directly or indirectly, with T cells, less is known about APCs effects on the immune system. For example, in a murine model of experimental autoimmune encephalitis, administration of APC or hirudin, an anticoagulant, reduced disease severity, and studies using APC mutants indicated that both anticoagulant activity and cell signaling activity were important for optimal effects (see, for example, Han et al., 2008, Nature, 451:1076-81). In addition, in vitro treatment of murine T cells with APC decreases the amount of IL17 produced following stimulation by CD3/CD28 (see, for example, Han et al., 2008, Nature, 451:1076-81). Further, APC showed immunosuppressive effects on murine dendritic cell populations in culture (see, for example, Kerschen et al., 2010, J. Clin. Invest., 120:3167-78), and APC suppressed markers of autoimmunity in a murine model of systemic lupus nephritis (see, for example, Lichtnekert et al., 2011, J. Immun., 187:3413-21). In a murine model of autoimmune diabetes, APC suppressed development of diabetes, which was attributed, in part, to the ability of APC to increase the frequency and function of regulatory T cells (see, for example, Xue et al., 2012, J. Biol. Chem., 287:16356-64). Mechanistically, APC's apparent therapeutic benefits in autoimmune encephalomyelitis required both anticoagulant and cell signaling activities. For APCs therapeutic benefits in endotoxic or bacterial sepsis, however, APCs cell signaling actions, but not its anticoagulant actions, are critical. There are no reports in the literature that describe the action, beneficial or otherwise, of APC or signaling-selective APC variants or mutants on GVHD or on T cells in the context of GVHD.

A number of APC variants or mutants are known in the art. In some instances, an APC variant or mutant exhibits predominantly or only one of the functions associated with APC. See, for example, Gale et al. (1997, Protein Sci., 6:132-40); Gale et al. (2002, J. Biol. Chem., 277:28836-40); Mosnier & Griffin (2003, Biochem. J., 373:65-70); Mosnier et al. (2004, Blood, 104:1740-4); Mosnier et al. (2012, Blood, 120:5237-46); Wildhagen et al. (2011, Thrombos. Haemost., 106:1034-45); Quinn et al. (2015, Biochem. Soc. Transact., 43:691-5); Griffin et al. (2015, Blood, 125:2898-907); Griffin et al. (2016, Thromb. Res., 141 Suppl 2:S62-64); Griffin et al. (2016, Atheroscl. Thrombos. Vasc. Biol., in press); WO 2004/056309; and WO 2005/007820. APC variants or mutants are known that exhibit predominantly the cell signaling activities of APC; these APC variants or mutants can be referred to as “signaling-selective” or “signal-selective” APC variants or mutants. Signaling-selective APC variants or mutants refer to APC variants or mutants in which one or more of the cell signaling activities is substantially retained (e.g., at least about 80%, 85%, 90%, 95% or more relative to wild type activity) but the anti-thrombotic or anticoagulant activity has been reduced (e.g., by at least about 25%, 30%, 35%, 40%, 45%, 50%, 75% or more relative to wild type activity) or eliminated (or essentially eliminated; e.g., reduced by at least about 90%, 91%, 92%, 93%, 94%, 95%, 96%, 97%, 98%, 99%, or 100% relative to wild type activity).

Several signaling-selective APC variants or mutants have been generated; these signaling-selective APC variants or mutants have been designated 3A-APC (APC with three sequential lysine residues replaced with three sequential alanine residues; APC with mutation Lys191-193Ala (residue numbering relative to mature human protein C)) and “5A-APC” (APC with mutation Lys191-193Ala and Arg229-230Ala (residue numbering relative to mature human protein C)). See, for example, WO 2008/055145; and WO 2008/073603. 3A-APC currently is in Phase 2 clinical trials for treating ischemic stroke. See, for example, Lyden et al. (2013, Curr. Pharm. Design, 19:7479-85).

Methods of Treating or Preventing GVHD with APC

This disclosure describes methods of treating or preventing graft-versus-host disease (GVHD) in a subject by administering APC or a variant or mutant thereof to the subject. APC or a variant or mutant thereof can be administered to a subject prior to the subject receiving a transplant. Additionally or alternatively, APC or a variant or mutant thereof can be administered to the subject concurrently with the transplant and/or at any time after they have received a transplant. As used herein, “transplant” typically refers to a blood or a bone marrow transplant such as, for example, an allogeneic blood or bone marrow transplant. Also additionally or alternatively, donor cells (e.g., donor T cells) can be contacted with APC or a variant or mutant thereof ex vivo prior to transplantation into the recipient.

APC or a variant or mutant thereof can be formulated with a pharmaceutically acceptable carrier for delivery to an individual in a therapeutically-effective amount. The particular formulation and the therapeutically-effective amount are dependent upon a variety of factors including, but not limited to, the route of administration, the dosage and dosage interval of the APC or a variant or mutant thereof, the sex, age, and weight of the subject being treated, and the severity of the GVHD.

As used herein, “pharmaceutically acceptable carrier” is intended to include any and all excipients, solvents, dispersion media, coatings, antibacterial and anti-fungal agents, isotonic and absorption delaying agents, and the like, compatible with administration. The use of such media and agents for pharmaceutically acceptable carriers is well known in the art. Except insofar as any conventional media or agent is incompatible with a compound, use thereof is contemplated.

Pharmaceutically acceptable carriers are well known in the art. See, for example Remington: The Science and Practice of Pharmacy, University of the Sciences in Philadelphia, Ed., 21^{st} Edition, 2005, Lippincott Williams & Wilkins; and The Pharmacological Basis of Therapeutics, Goodman and Gilman, Eds., 12^{th} Ed., 2001, McGraw-Hill Co. Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, N.Y.

A pharmaceutical composition that includes a compound as described herein is typically formulated to be compatible with its intended route of administration. Suitable routes of administration include, for example, oral, rectal, topical, nasal, pulmonary, ocular, intestinal, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

For intravenous injection, for example, the composition may be formulated as an aqueous solution using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, optionally containing penetration enhancers, which are known in the art. For oral administration, a compound can be formulated in liquid or solid dosage forms, and also formulation as an instant release or controlled/sustained release formulations. Suitable dosage forms for oral ingestion by an individual include tablets, pills, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which can include fillers, disintegrants, binders (dry and wet), dissolution retardants, lubricants, glidants, anti-adherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (e.g., dextrose, sucrose, lactose), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. When a capsule is preferred over a tablet, the drug powder, suspension, or solution thereof can be delivered in a compatible hard or soft shell capsule.

APC or variants or mutants thereof can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and the use of complexing agents. For administration by inhalation (e.g., via the mouth or nose), compounds can be delivered in the form of a solution, suspension, emulsion, or semisolid aerosol from pressurized packs, or a nebuliser, usually with the use of a propellant, e.g., halogenated carbons.

Compounds described herein also can be formulated for parenteral administration (e.g., by injection). Such formulations are usually sterile and, can be provided in unit dosage forms, e.g., in ampoules, syringes, injection pens, or in multi-dose containers, the latter usually containing a preservative. The formulations may take such forms as suspensions, solutions, or emulsions in oily or aqueous vehicles, and may contain other agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Polymers such as poly(lactic acid), poly(glycolic acid), or copolymers thereof, can serve as controlled or sustained release matrices, in addition to others well known in the art. Other delivery systems may be provided in the form of implants or pumps.

APC or variants or mutants thereof can be administered at least once a day (e.g., at least twice a day, at least three times a day, or more) to a subject suffering from GVHD or at risk of developing GVHD. APC or variants or mutants thereof can be administered to a subject for a short period of time (e.g., for one or a few days, for one or a few weeks), or APC or variants or mutants thereof can be administered chronically (e.g., for several weeks, months or years) to a subject suffering from GVHD or at risk of developing GVHD.

APC or a variant or mutant thereof can be administered in a therapeutically effective amount to a subject suffering from GVHD. Typically, a therapeutically effective amount is an amount that imparts beneficial effects without inducing any adverse effects. Toxicity and therapeutic efficacy of APC or a variant or mutant thereof can be determined by standard pharmaceutical procedures in cell cultures or experimental animals, e.g., by determining the LD₅₀ (the dose lethal to 50% of the population), the ED₅₀ (the dose therapeutically effective in 50% of the population), and/or the LD₅₀/ED₅₀ ratio (the therapeutic index, expressed as the dose ratio of toxic to therapeutic effects). The method of any of the proceeding claims, wherein the APC or the variant or mutant thereof is administered to the subject at a dose of about 0.01 mg to about 0.6 mg of the APC or the variant or mutant thereof per kilogram (kg) of the subject (e.g., about 0.05 to about 0.5 mg APC/kg; about 0.075 to about 0.3 mg APC/kg; about 0.05 to about 0.2 mg APC/kg; about 0.1 to about 0.5 mg/kg; about 0.2 to about 0.4 mg APC/kg; about 0.1 mg APC/kg; about 0.2 mg APC/kg; about 0.3 mg APC/kg). For example, suitable ranges of the APC or the variant or mutant thereof include, without limitation, about 0.12 mg to about 0.24 mg per kilogram (kg) of the subject.

As used herein, “treating” refers to reversing, alleviating, or inhibiting the progression of GVHD, or one or more symptoms associated with GVHD and “preventing” refers to avoiding or precluding the development of GVHD or one or more of the symptoms associated with GVHD. It would be understood that the particular therapeutic endpoint(s) that determines whether or not treatment has been achieved (e.g., whether or not a patient has been treated) will depend upon how the GVHD manifests itself (e.g., the tissue or organs affected, the severity or acuteness of the disease, or the coexistence of more than one disease) in each subject. For examples of therapeutic and clinical guidelines for GVHD, see, for example, Lee et al. (2015, Biol. Blood Marrow Transplant., 21:984-999); Jagasia et al. (2015, Biol. Blood Marrow Transplant., 21:389-401); and Miklos et al. (2017, Blood, doi: 10.1182/blood-2017-07-793786).

Briefly, clinical cGVHD can involve not only classical acute GVHD (aGVHD) epithelial target tissues (e.g., GI tract, liver, skin, lung) but any other organ system including, without limitation, oral, esophageal, musculoskeletal, joint, fascial, hair and nails, ocular, lymphohematopoietic system and genital tissues. Eight organ systems (i.e., skin, mouth, eyes, gastrointestinal tract, liver, lungs, genital tract and fasciae/joints) evaluated for diagnosis are scored (range 0-3) for individual organ system severity and summed to calculate global cGVHD severity. Primary efficacy endpoints are best overall cGVHD response rate, which is defined as the proportion of all subjects who achieve a complete response (CR) or partial response (PR) (based on the 2014 NIH Consensus Panel). All subjects who have at least one response assessment are considered response-evaluable. Secondary efficacy end points include sustained response of ≥20 weeks, changes in corticosteroid requirement over time, and change in the Lee cGVHD Symptom Scale (self-reported). A decrease by ≥7 points is considered clinically meaningful and relates to improved quality of life.

### Transgenic Animals

Transgenic animals are animals that carry a genetically-inheritable change in their genome. This disclosure provides transgenic animals (i.e., non-human animals) whose genome includes a mutation at Arg41 or Arg46 in the protease activated receptor 1 (PAR1) sequence, which is encoded by the murine gene, F2r. A transgenic non-human animal can include, without limitation, a mouse, a rat, a guinea pig, a sheep, a zebrafish, a pig, a dog, or a primate. Animals that carry a mutation in Arg41 in PAR1 typically exhibit a phenotype of lower than expected homogenous offspring, similar to the situation for PAR1-knockout mice where breeding of heterozygotes yields only 10% to 15% of homozygous altered mice rather than the expected 25%. Animals that carry a mutation in Arg46 in PAR1 typically do not have a detectable phenotype. As described herein, such transgenic animals can be used to evaluate the mechanisms by which APC or variants or mutants thereof trigger cell signaling (e.g., protective cell signaling; harmful or damaging cell signaling).

Methods of making a transgenic animal that carries a mutation in either the Arg41 or the Arg46 in PAR1 are known. For example, a mutation can be introduced into an animal at an embryonic stage, preferably the one cell stage, or fertilized egg stage, and generally not later than about the 8-cell stage. The zygote or embryo is then carried to term in a pseudo-pregnant female that acts as a surrogate mother. A pseudo-pregnant female refers to a female in estrus who has mated with a vasectomized male; she is therefore competent to receive embryos but does not contain any fertilized eggs. In order to achieve stable inheritance of the introduced nucleic acid, the mutation must occur in a cell type that can give rise to functional germ cells (i.e., sperm or oocytes). Two animal cell types that can form germ cells and into which DNA can be readily introduced or manipulated are fertilized egg cells and embryonic stem (ES) cells. Methods of producing transgenic animals using zygote injection is described, for example, in U.S. Pat. No. 4,736,866, and methods of producing transgenic animals using ES cells is described, for example, in U.S. Pat. Nos. 4,396,601 and 4,497,796.

Nuclear transplantation also can be used to generate non-human transgenic animals. For example, fetal fibroblasts can be genetically modified such that they contain a mutation in Arg41 or Arg46 of PAR1, and then such cells can be fused with enucleated oocytes. After activation of the oocytes, the eggs can be cultured to the blastocyst stage and implanted into a recipient. See, for example, Cibelli et al. (1998, Science, 280:1256-58). Adult somatic cells including, for example, cumulus cells and mammary cells, can be used to produce animals such as mice and sheep, respectively. See, for example, Wakayama et al. (1998, Nature, 394:369-74); and Wilmut et al. (1997, Nature, 385:810-13). Nuclei can be removed from genetically modified adult somatic cells, and transplanted into enucleated oocytes. After activation, the eggs can be cultured to the 2- to 8-cell stage, or to the blastocyst stage, and implanted into a suitable recipient. See, for example, Wakayama et al., supra.

Mutations can be introduced into genomic nucleic acid using known methods. For example, mutations can be introduced using homologous recombination with a construct (e.g., a recombinant adeno-associated virus (rAAV)) that contains a PAR1 sequencing carrying the particular mutation (i.e., at Arg41 or at Arg46). Alternatively, genomic DNA can be modified using any number of genome editing tools such as, without limitation, zinc finger nucleases, TALENs, or CRISPR/Cas.

The mutation can be detected in weanling animals (4-5 weeks) using methods known to those of skill in the art (e.g., hybridization, PCR amplification), and standard physiological tests also can be performed on such transgenic animals, such as a complete blood count (CBC) and glucose uptake. The animals used as a source of fertilized eggs or embryonic stem (ES) cells, i.e., the host animal, can be any animal, although generally the host animal is one that lends itself to multigenerational studies. Another characteristics of a host animal includes longevity to the extent that there is sufficient time for observable physiological and/or pathological changes to occur in the animal.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Effects of Activated Protein C (APC) on Pulmonary Function

To determine whether APC has in vivo immune suppressive activity, mice were conditioned with high dose cyclophosphamide and total body irradiation followed by MHC-disparate bone marrow and splenocytes. This results in an excellent murine animal model of multi-organ system chronic graft versus host disease (cGVHD) with bronchiolitis obliterans (BO) and fibrosis. Mice were administered recombinant murine APC (6 μg/dose; approximately 0.24 mg/kg) or vehicle intraperitoneally once daily from day 28 through day 56. Three forms of APC were studied: wild type (WT)-APC and two APC variants with greatly reduced anticoagulant activity but substantial cell signaling activity, 3A-APC (mutation of Lys191-193Ala (human protein C numbering) and 5A-APC (mutation of Lys191-193Ala plus Arg229-230Ala (human protein C numbering)).

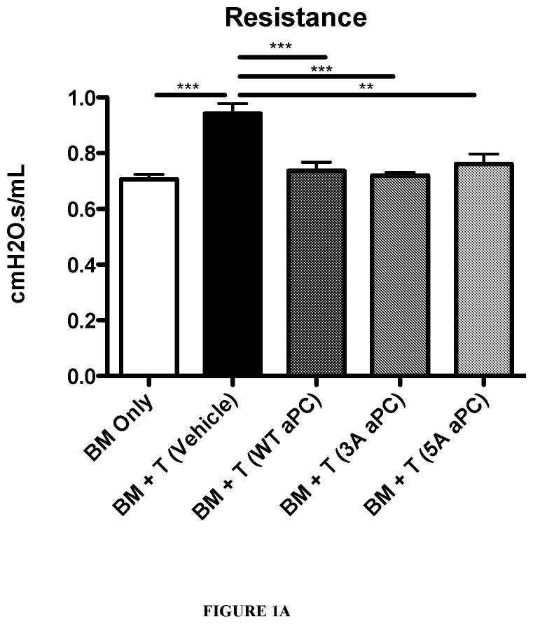

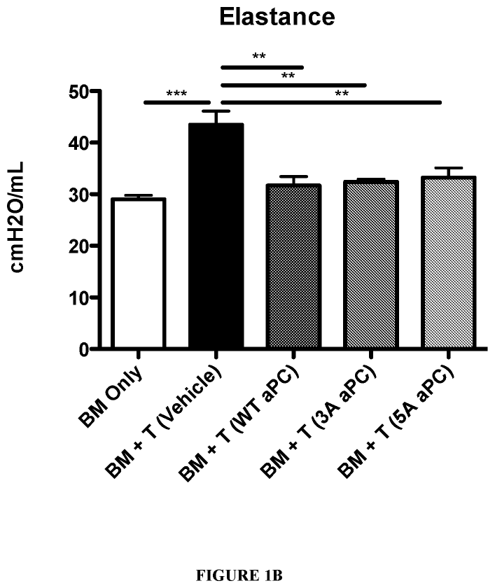

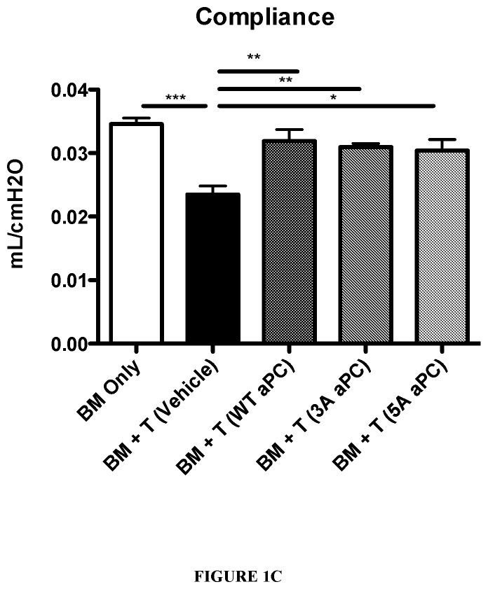

Pulmonary function tests (resistance, elastance, and compliance; FIGS. 1A, 1B and 1C, respectively), which are indicative of cGVHD BO, were markedly improved by WT-APC and by both anticoagulant defective mutants, 3A-APC and 5A-APC. Thus, APC therapy reduced lung injury and improved lung function.

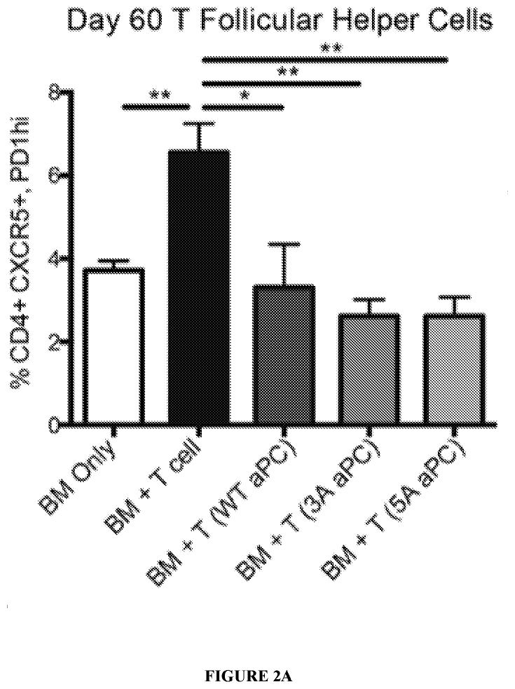

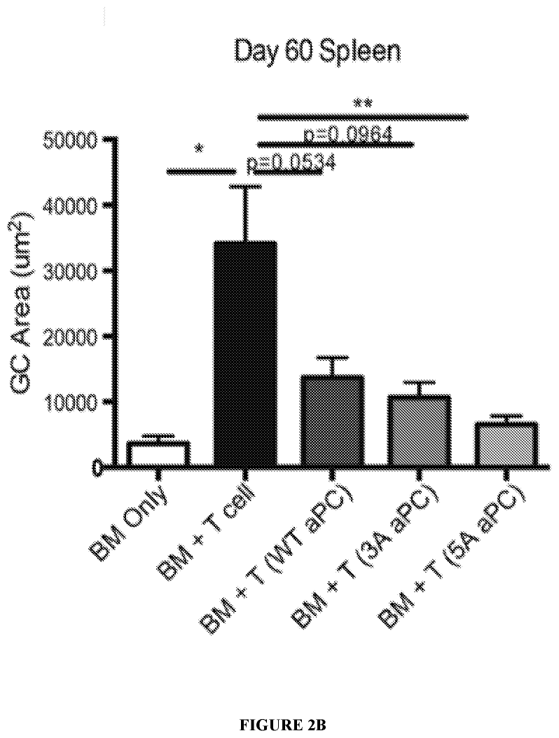

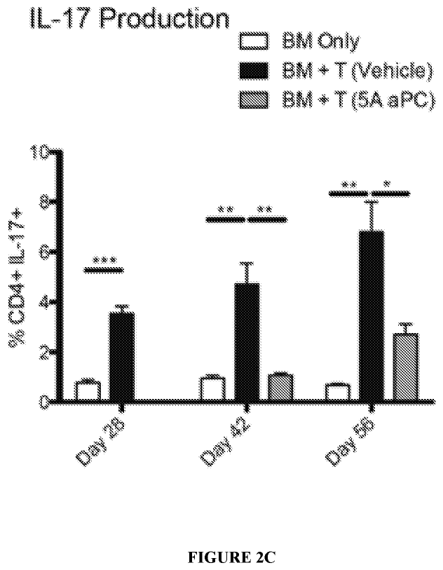

APC treatment also significantly reduced splenic germinal center (GC) size (FIG. 2B) as well as T follicular helper cell (CD4+CXCR5+PD1hi) (FIG. 2A) and Th17 frequencies (FIG. 2C), both of which are critical for GC formation and subsequent fibrosis. These data indicated, therefore, that WT-APC and its variants, 3A-APC and 5A-APC, significantly reduced pulmonary fibrosis.

Together, these data demonstrated that APC and variants thereof significantly reduced the pulmonary pathology in cGVHD and IL-17-positive T cells, and that this reduction was primarily due to the signaling activity of APC, rather than the anticoagulant activity of APC.

### Example 2—Identification of APC Activity Responsible for Reducing GVHD

The lack of difference in efficacy between WT and the signaling-selective variants suggests APC-induced signaling, not anticoagulant activity, is primarily responsible for reduction of pulmonary pathology in cGVHD. To determine if APCs benefits for cGVHD might require PAR1 cleavage at Arg41 (i.e., to induce G-protein-dependent effects) or PAR1 cleavage at Arg46 to induce beta-arrestin-dependent biased signaling, genetically modified mice were used. ES cells and homologous recombination were used to generate C57BL/6 mice strains carrying the PAR1 mutation of either Arg41Gln (R41Q) or Arg46Gln (R46Q). Then donor T cells were prepared from wild type (WT) control mice and from each genetically altered strain (i.e., from mice carrying WT-PAR1, QQ41-PAR1, or QQ46-PAR1) and were used to induce cGVHD.

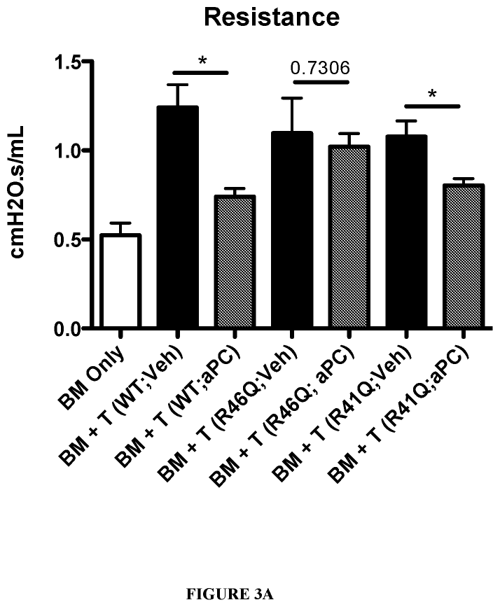

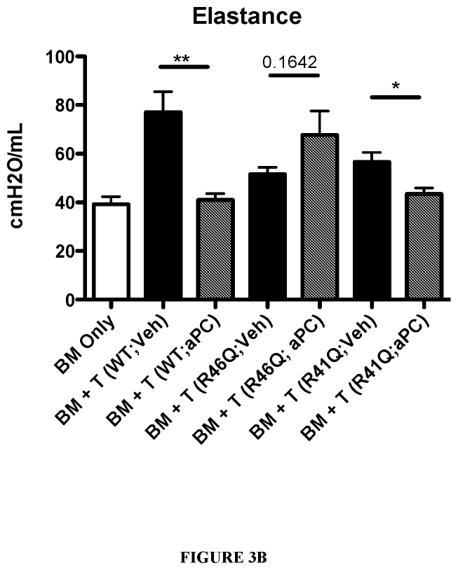

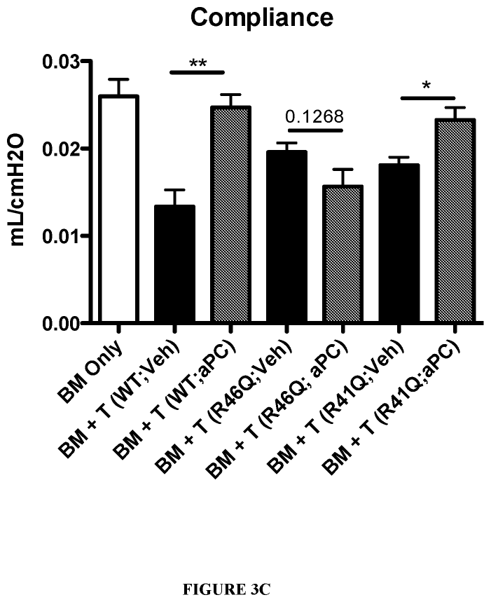

Results demonstrated that 5A-APC reduced cGVHD for transplanted T cells carrying WT-PAR1 and QQ41-PAR1 (FIG. 3), but 5A-APC was completely ineffective in reducing cGVHD for transplanted QQ46-PAR1 T cells, indicating that Arg46 in PAR1 in donor T cells is essential for the benefits observed with 5A-APC. Since APC's cleavage at Arg46 is key for triggering PAR1 biased signaling, it was inferred that APC's benefits for cGVHD require, at least in part, direct biased activation of PAR1 on donor T cells by APC and that this signaling precludes sufficient IL17 generation and GC formation to cause cGVHD.

### Example 3—Summary of Results

In summary, daily therapy using recombinant APC was highly effective in treating ongoing cGVHD in a BO model. Studies using signaling-selective APC mutants and donor T cells obtained from novel PAR1-genetically modified mice suggest that the mechanisms responsible for cGVHD suppression involve APC-induced signaling activity and not APC's anticoagulant activity. Such signaling resulted in suppression of Tfollicular helper cells and Th17. Given the safety and efficacy record of 3A-APC in Phase 1 clinical studies, these data strongly support the consideration of APC, of 3A-APC or 5A-APC, or of signaling-selective variants thereof for treatment of cGVHD with BO manifestations that have been refractory to other therapies. Based on the results herein, treatment with APC or a variant or mutant thereof (e.g., of donor T cells prior to transplantation or of a recipient subject prior to, during or after receiving a transplant) favorably alters the development and differentiation of donor T cells in a manner that reduces GVHD without the risk of bleeding diatheses.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of treating or preventing graft-versus-host disease (GVHD) in a subject, comprising:
administering a therapeutically effective amount of protein C or variant or mutant thereof to the subject.

2. The method of claim 1, wherein the protein C or variant or mutant thereof is an activated protein C (APC) or variant or mutant thereof.

3. The method of claim 2, wherein the APC variant or mutant thereof is a signaling-selective APC variant or mutant.

4. The method of claim 2, wherein the APC variant or mutant is APC with a Lys191-193Ala mutation (3A-APC).

5. The method of claim 2, wherein the APC variant or mutant is APC with Lys191-193Ala and Arg229-230Ala mutations (5A-APC).

6. The method of claim 1, wherein the protein C or variant or mutant thereof is administered to the subject at least once a day.

7. The method of claim 1, wherein the protein C or variant or mutant thereof is administered to the subject at a dose of about 0.01 mg to about 0.6 mg of the protein C or variant or mutant thereof per kilogram (kg) of the subject.

8. The method of claim 1, wherein the protein C or variant or mutant thereof is administered to the subject at a dose of about 0.12 mg to about 0.24 mg of the protein C or variant or mutant thereof per kilogram (kg) of the subject.

9. The method of claim 1, wherein the therapeutically-effective amount of protein C or variant or mutant thereof is about 0.01 mg to about 0.6 mg of the protein C or variant or mutant thereof per kilogram (kg) of the subject.

10. The method of claim 1, wherein the therapeutically-effective amount of protein C or variant or mutant thereof is about 0.12 mg to about 0.24 mg of the protein C or variant or mutant thereof per kilogram (kg) of the subject.

11. The method of claim 1, wherein the protein C or variant or mutant thereof is administered intraperitoneally.

12. The method of claim 1, wherein the administering occurs prior to the subject receiving a bone marrow transplant.

13. The method of claim 1, wherein the administering occurs coincidentally with the subject receiving a bone marrow transplant.

14. The method of claim 1, wherein the administering occurs after the subject has received a bone marrow transplant.

15. The method of claim 1, wherein GVHD is treated in the subject when the GVHD or one or more symptoms associated with the GVHD is reversed, alleviated or inhibited.

16. The method of claim 1, wherein GVHD is prevented in the subject when the GVHD or one or more symptoms associated with GVHD is avoided or precluded.

17. The method of claim 1, wherein the subject has received a hematopoietic stem cell transplant.

18. The method of claim 17, wherein the hematopoietic stem cell transplant is an allogeneic hematopoietic stem-cell transplant.

19. The method of claim 17, wherein the hematopoietic stem cell transplant is a bone marrow transplant.

20. The method of claim 1, wherein the GVHD is chronic GVHD.

21. The method of claim 1, wherein the GVHD is acute GVHD.

22. The method of claim 1, wherein the subject further suffers from multi-organ system disease with bronchiolitis obliterans (BO).

23. The method of claim 1, wherein the subject further suffers from fibrosis.

24. A method of treating or preventing graft-versus-host disease (GVHD) in a subject, comprising:
contacting donor T cells with a therapeutically effective amount of protein C or variant or mutant thereof.

25. The method of claim 24, wherein the protein C or a variant or mutant thereof is an activated protein C (APC) or variant or mutant thereof.

26. The method of claim 24, wherein the donor T cells are in blood.

27. The method of claim 24, wherein the donor T cells are in bone marrow.

28. The method of claim 24, wherein the donor T cells are contacted ex vivo.
